(19) United States
(12) Patent Application Publication
(10) Pub. No.: US 2024/0260998 A1
(43) Pub. Date: Aug. 8, 2024

(54) COUPLING SLEEVE FOR BONE POWER TOOL

(71) Applicant: OrthoPediatrics Corp., Warsaw, IN (US)

(72) Inventors: Camden L. Fox, Fort Wayne, IN (US); Richard T. Detlefsen, Warsaw, IN (US)

(21) Appl. No.: 18/435,525

(22) Filed: Feb. 7, 2024

Related U.S. Application Data
(60) Provisional application No. 63/483,910, filed on Feb. 8, 2023.

(51) Int. Cl. A61B 17/70; A61B 17/00

(52) U.S. Cl. CPC A61B 17/702; A61B 2017/00477

(57) ABSTRACT

A coupling sleeve for a bone power tool and a bone anchor is provided. The coupling sleeve includes a proximal collar, a distal collar, and a central portion. The proximal collar defines a first opening that is configured to interface with a bone power tool. The distal collar defines a second opening that is configured to interface with a retention feature for a pedicle screw. The central portion extends between the proximal collar and the distal collar. The proximal collar, the distal collar, and the central portion cooperate to define a passageway between the first opening and the second opening.

### REFERENCE TO RELATED APPLICATION

This application claims priority of U.S. Pat. App. No. 63/483,910, entitled “Coupling Sleeve for Bone Power Tool,” filed Feb. 8, 2023, the disclosure of which is incorporated by reference herein in its entirety.

### TECHNICAL FIELD

The apparatus described below generally relates to a coupling sleeve that facilitates attachment of a set screw to a bone anchor for use in an orthopedic procedure, such as spinal alignment. In particular, the coupling sleeve can facilitate rotational coupling between a bone power tool and a bone anchor to allow for driving of the set screw thereto.

### BACKGROUND

Spinal fixation systems can be used to surgically fix, adjust, and/or align the spinal column. One type of a spinal fixation system employs a spinal rod for supporting the spine and fixing, adjusting, and/or aligning all or portions of the spinal column into a desired orientation. Attachment of the spinal rod to the spinal column has typically been achieved using a variety of bone anchors. These bone anchors typically require a set screw that facilitates attachment of the spinal rod to the bone anchors.

### BRIEF DESCRIPTION OF THE DRAWINGS

Various embodiments will become better understood with regard to the following description, appended claims and accompanying drawings wherein:

FIG. 1 is an upper isometric exploded view depicting a torque limiting attachment, a driver bit, a coupling sleeve, a retention tip, a set screw, a tulip head, and a pedicle screw, in accordance with one embodiment;

FIG. 2 is an upper isometric assembled view depicting the torque limiting attachment, the driver bit, the coupling sleeve, the retention tip, the set screw, the tulip head, and the pedicle screw of FIG. 1;

FIG. 3 is an upper isometric view of the coupling sleeve of FIG. 1;

FIG. 4 is a lower isometric view of the coupling sleeve of FIG. 3;

FIG. 5 is an upper isometric view of the retention tip of FIG. 1;

FIG. 6 is a lower isometric view of the retention tip of FIG. 5;

FIG. 7 is a cross-sectional view taken along the line 7-7 in FIG. 6;

FIG. 8 is an upper isometric exploded view depicting a torque limiting attachment, a driver bit, a coupling sleeve, a rod reducer, a set screw, a tulip head, and a pedicle screw, in accordance with another embodiment; and

FIG. 9 is an upper isometric assembled view depicting the torque limiting attachment, the driver bit, the coupling sleeve, the rod reducer, the set screw, the tulip head, and the pedicle screw of FIG. 8.

### DETAILED DESCRIPTION

Various non-limiting embodiments of the present disclosure will now be described to provide an overall understanding of the principles of the structure, function, and use of the apparatuses, systems, methods, and processes disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

The examples discussed herein are examples only and are provided to assist in the explanation of the apparatuses, devices, systems and methods described herein. None of the features or components shown in the drawings or discussed below should be taken as mandatory for any specific implementation of any of these the apparatuses, devices, systems or methods unless specifically designated as mandatory. For ease of reading and clarity, certain components, modules, or methods may be described solely in connection with a specific figure. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Described herein are example embodiments of a coupling sleeve that can be associated with a bone power tool and a retention fixture (e.g., a reduction rod or a retention tip) that cooperate to facilitate implantation of a bone anchor (e.g., hooks, screws, etc.) into a patient's bone during orthopedic procedures such as, for example, spinal fixation. In the illustrated embodiments, a coupling sleeve is disclosed as being in association with different retention fixtures that engage with a bone anchor (e.g., a tulip head and associated pedicle screw) to teach the features of the coupling sleeve and how the coupling sleeve engages and/or works with such illustrative bone power tools, retention fixtures and bone anchors, but the disclosure is not to be limited only to the bone power tools, retention fixtures, and bone anchors illustrated herein.

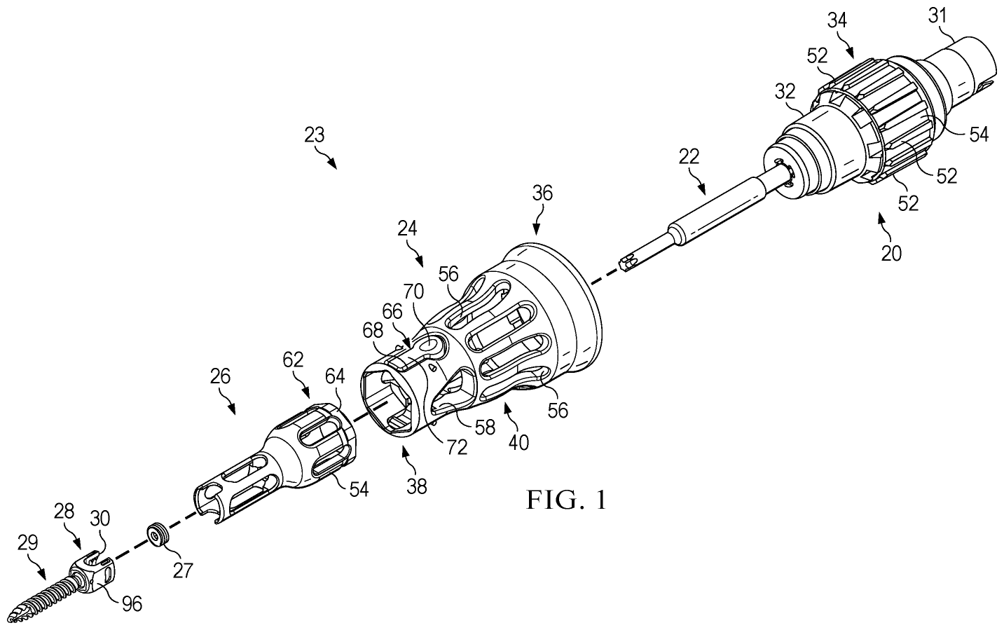

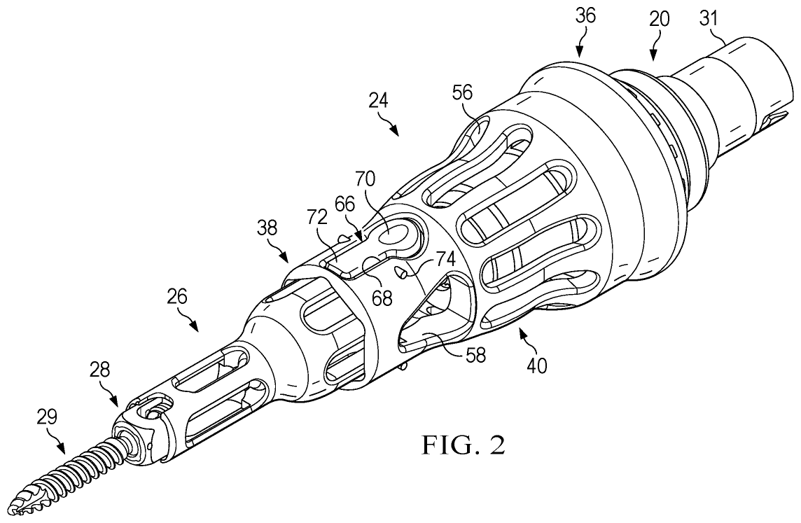

Embodiments are hereinafter described in detail in connection with the views and examples of FIGS. 1-9, wherein like numbers indicate the same or corresponding elements throughout the views. A torque limiting attachment 20, a driver bit 22, and a coupling assembly 23 that includes a coupling sleeve 24 and a retention tip 26 are generally depicted in FIGS. 1 and 2. The torque limiting attachment 20 can support the driver bit 22 for driving a set screw 27 (FIG. 1). The set screw 27 can be associated with a tulip head 28 that supports a pedicle screw 29 or other type of bone anchor. The tulip head 28 can include a threaded interface 30 that is configured to allow the set screw 27 to be threaded onto the tulip head 28. The torque limiting attachment 20 can include a driveshaft sleeve 31 and a rotary chuck 32. The driveshaft sleeve 31 can house a driveshaft (not shown) that is operably coupled to the rotary chuck such that rotation of the driveshaft causes the rotary chuck 32 to thereby rotate the driver bit 22. The driveshaft can be operably coupled with a bone power tool (not shown), such as a battery powered handheld drill driver, that supplies rotary power to the driveshaft. The rotary chuck 32 can selectively grasp the driver bit 22 and can allow for different driver bits (e.g., 22) to be installed in the rotary chuck 32 to interface with different set screws (e.g., 27). The torque limiting attachment 20 can also include a stationary collar 34 that surrounds the driveshaft and the rotary chuck 32 near the center of the torque limiting attachment 20. The stationary collar 34 can be fixed relative to the driveshaft and the rotary chuck 32 such that the driveshaft and the rotary chuck 32 rotate relative to the stationary collar 34.

The coupling sleeve 24 can be coupled with the torque limiting attachment 20 and the retention tip 26 can be coupled with the coupling sleeve 24 to accommodate the tulip head 28. The coupling sleeve 24 and the retention tip 26 can be hollow to allow the driver bit 22 to extend from the torque limiting attachment 20, through the coupling sleeve 24 and the retention tip 26 and into engagement with the set screw 27 at the tulip head 28. The coupling sleeve 24 can include a proximal collar 36, a distal collar 38, and a central portion 40 disposed therebetween. As illustrated in FIG. 2, the torque limiting attachment 20 can be installed in the proximal collar 36 and the retention tip 26 can be installed in the distal collar 38 to hold the tulip head 28 and the pedicle screw 29 in place relative to the driver bit 22. The coupling sleeve 24 can be formed as a unitary one-piece construction, such as, for example, through a molding (e.g., casting), extrusion, machining, or additive manufacturing process. In one embodiment, the coupling sleeve 24 can be formed of stainless steel but any of a variety of suitable alternative materials are also contemplated.

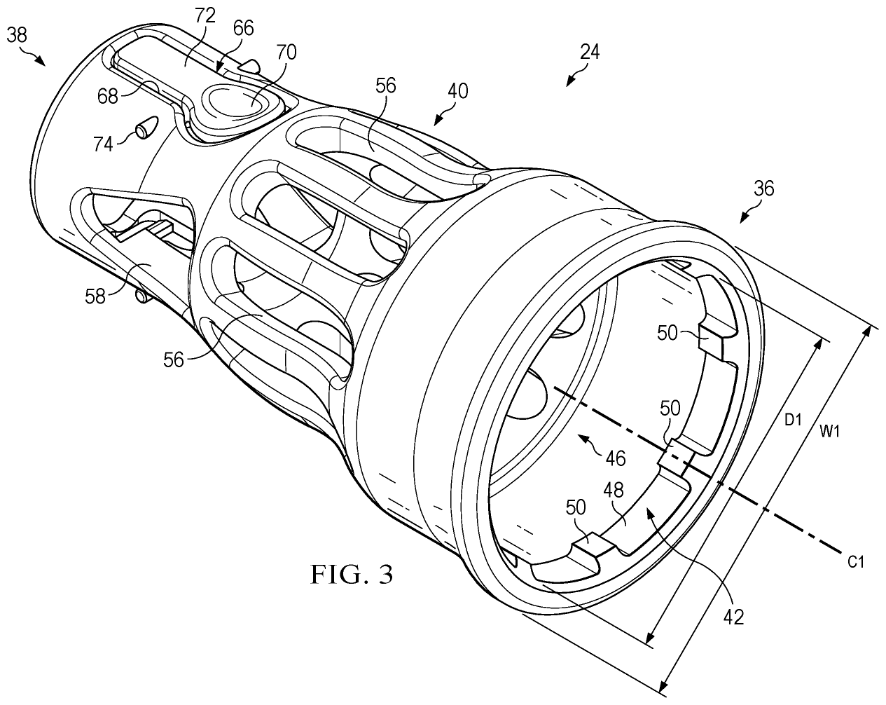

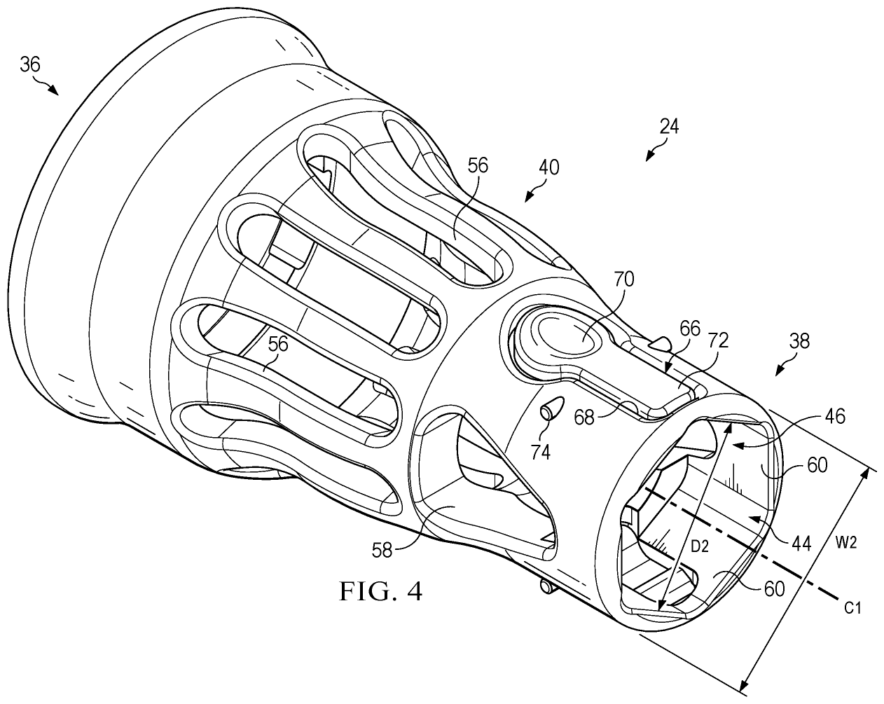

Referring now to FIGS. 3 and 4, the proximal collar 36 can define an opening 42 (FIG. 3) and the distal collar 38 can define an opening 44 (FIG. 4). The coupling sleeve 24 can define a passageway 46 that extends through the proximal and distal collars 36, 38, through the central portion 40 and between the openings 42, 44. The coupling sleeve 24 can define a centerline C1 that is central to the openings 42, 44 and the passageway 46. When the retention tip 26 is installed on the coupling sleeve 24, the retention tip 26 and the tulip head 28 can be provided along the centerline C1. When the torque limiting attachment 20 is installed on the coupling sleeve 24, the driver bit 22 can extend through the torque limiting attachment 20 and can be substantially aligned with the centerline C1 such that the driver bit 22 is able to engage the set screw 27 to allow the bone power tool to rotate the set screw 27 via the driver bit 22.

As illustrated in FIG. 3, the opening 42 of the proximal collar 36 can be formed by an interior wall 48 and can have a cross-sectional shape that is complimentary to the cross-sectional shape of the stationary collar 34. For example, the interior wall 48 can be substantially cylindrical shaped and can include a plurality of inner splines 50 that extend radially inwardly (e.g., towards the centerline C1). The inner splines 50 can be configured to mate with outer splines 52 that extend radially outwardly from an outer wall 54 of the stationary collar 34 (FIG. 1). When the torque limiting attachment 20 is installed in the proximal collar 36, the mated interface between the inner and outer splines 50, 52 can rotationally couple the coupling sleeve 24 to the stationary collar 34 to prevent rotation therebetween while also allowing the torque limiting attachment 20 to slide axially relative to the coupling sleeve 24 (e.g., along the centerline C1). As such, the coupling sleeve 24 can be slid onto/from the torque limiting attachment 20 to facilitate installation/removal and can slide relative to the stationary collar 34 during actuation of the set screw 27 to allow the driver bit 22 to travel longitudinally with the set screw 27. It is to be appreciated that the stationary collar 34 and the opening 42 can have any of a variety of suitable alternative complimentary cross-sectional shapes, such as polygonal shapes (e.g., triangular, square, or hexagonal). It is also to be appreciated that any of a variety of other suitable coupling arrangements are contemplated, such as, for example, a cross pin connection, a bayonet connection, a keyway, a split collar and clamp arrangement, or a threaded connection.

As illustrated in FIGS. 3 and 4, the central portion 40 of the coupling sleeve 24 can define a plurality of windows 56 and the distal collar 38 can define a pair of windows 58 that can reduce the overall weight of the coupling sleeve 24 and in some instances can serve as a viewing window (i.e., the windows 58) into the passageway 46.

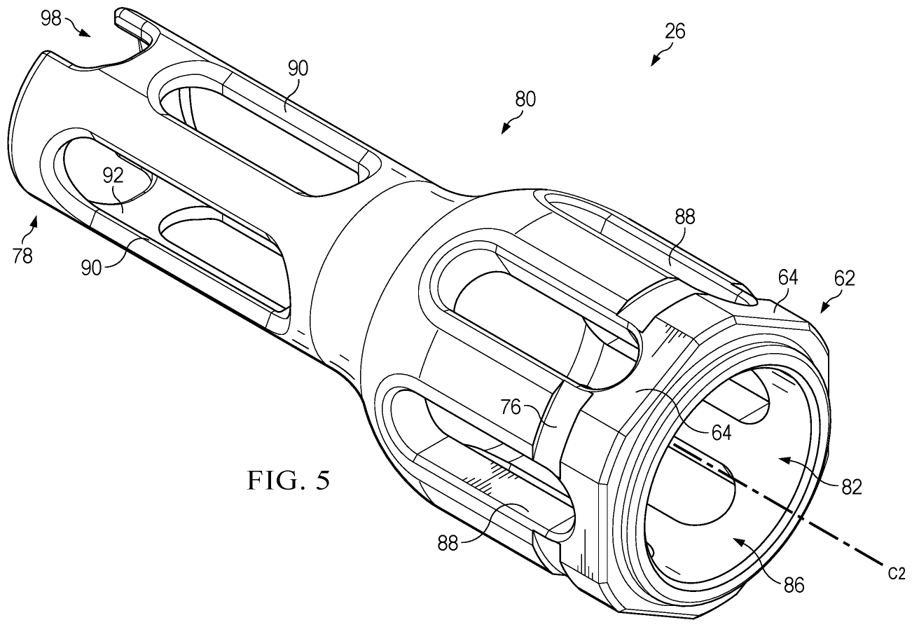

As illustrated in FIG. 4, the opening 44 of the distal collar 38 can be formed by an interior wall 60 and can have a cross-sectional shape that is complimentary to the cross-sectional shape of the retention tip 26 or otherwise configured to maintain rotational constraint between the interior wall 60 and the retention tip 26. For example, the interior wall 60 can be substantially hexagonally shaped. As illustrated in FIG. 5, the retention tip 26 can include a proximal end 62 that is configured for insertion into the opening 44 to facilitate coupling of the retention tip 26 to the coupling sleeve 24. The proximal end 62 can include an exterior wall 64 that has a similar hexagonal cross-sectional shape as the interior wall 60 and the opening 44 of the distal collar 38 of the coupling sleeve 24. The similarly shaped interior and exterior walls 60, 64 can interface with each other to allow the proximal end 62 to slide relative to the distal collar 38 (e.g., during installation and removal) while also preventing the retention tip 26 from rotating relative to the coupling sleeve 24. It is to be appreciated that the opening 44 and the proximal end 62 of the retention tip 26 can have any of a variety of suitable alternative complimentary cross-sectional shapes, such as other polygonal shapes (e.g., triangular, square) or a cylinder shape with splines.

Referring again to FIGS. 3 and 4, the opening 42 of the proximal collar 36 can have a diameter D1 (FIG. 3) and the opening 44 of the distal collar 38 can have a diameter D2 (FIG. 4). The diameter D1 can correspond to the overall diameter of the stationary collar 34 and the diameter D2 can correspond to the overall diameter of the retention tip 26. Since the diameter of the stationary collar 34 is greater than the diameter of the retention tip 26, the diameter D1 can be greater than the diameter D2 such that the passageway 46 is narrower at the opening 44 than at the opening 42 (i.e., the passageway 46 generally tapers inwardly in the direction of the opening 42). In one embodiment, the diameter D1 of the opening 42 can be about 1.5 times larger than the diameter D2 of the opening 44.

The relative difference between the diameters D1 and D2 allows the distal collar 38 to have a width W1 (FIG. 3) than is narrower than a width W2 (FIG. 4) of the proximal collar 36. As such, the central portion 40 of the coupling sleeve 24 can tapered inwardly in the direction of the distal collar 38 such that the overall profile of the coupling sleeve 24 has a substantially conical shape that is narrower at the distal collar 38 than the proximate collar 36. This conical shape can allow the coupling assembly 23 to easily pass through an incision when inserted into a surgical site for implanting the pedicle screw 29 into a desired location.

The size and cross-sectional shape of the opening 42 can be different from the size and shape of the opening 44 which can prevent the coupling sleeve 24 from being installed incorrectly. For example, when the coupling sleeve 24 is being installed and is in the proper orientation (with the proximal collar 36 positioned adjacent to the stationary collar 34 and the distal collar 38 positioned adjacent to the retention tip 26 (as shown in FIG. 1)), the stationary collar 34 is able to fit into the proximal collar 36 and the retention tip 26 is able to fit into the distal collar 38 when both are installed on the coupling sleeve 24. However, if the coupling sleeve 24 is provided in an inverted orientation, the stationary collar 34 and the proximal collar 36 are incapable of properly mating together and the retention tip 26 and the distal collar 38 are incapable of properly mating together which can indicate to a user that the coupling sleeve 24 is being installed incorrectly and needs to be inverted to facilitate proper installation.

Referring again to FIG. 4, the coupling sleeve 24 can include a pair of grasping members 66 that cooperate to facilitate selective retention of the retention tip 26 to the distal collar 38. The grasping members 66 can be disposed on opposite sides of the distal collar 38 and can reside in receptacles 68 defined by the distal collar 38. One of the grasping members 66 will now be described and can be understood to be representative of both of the grasping members 66. The grasping member 66 can include a pushbutton 70 and a latching arm 72 extending therefrom. The grasping member 66 can be pivotable about a pin 74 between a latched position (FIG. 4) and a released position (not shown) to facilitate selective retention of the retention tip 26 to the coupling sleeve. When the grasping member 66 is in the latched position, the latching arm 72 can extend into the passageway 46. When the retention tip 26 is installed in the coupling sleeve 24, the latching arm 72 can interface with a groove 76 (FIG. 5) defined on the proximal end 62 of the retention tip 26 to secure the retention tip 26 in place. When the grasping member 66 is pivoted into the released position, the latching arm 72 can be removed from the passageway 46 and the groove 76 (FIG. 5) to release the retention tip 26 from the coupling sleeve 24.

The grasping member 66 can be biased into the latching position by a spring (not shown). As such, when the retention tip 26 is being installed into the distal collar 38 of the coupling sleeve 24, the latching arm 72 can initially be pushed away from the latching position (by the grasping member 66 riding along the exterior wall 64) during insertion of the retention tip 26 into the coupling sleeve 24. Once the retention tip 26 is installed far enough that the groove 76 is aligned with the latching arm 72, the latching arm 72 can be automatically urged into the groove 76 (by the spring) to hold the retention tip 26 in place. To release the retention tip 26, a user can depress the pushbutton 70 which moves the latching arm 72 into the released position and away from the groove 76 thus freeing the retention tip 26 and allowing it to be removed from the coupling sleeve 24. It is to be appreciated that although two grasping members are shown, any quantity of grasping members are contemplated such as one or more than two. It is also to be appreciated that the coupling sleeve 24 and the retention tip 26 can be additionally or alternatively coupled together via threading, a friction fit, magnetic engagement, or any of a variety of suitable alternative coupling arrangements.

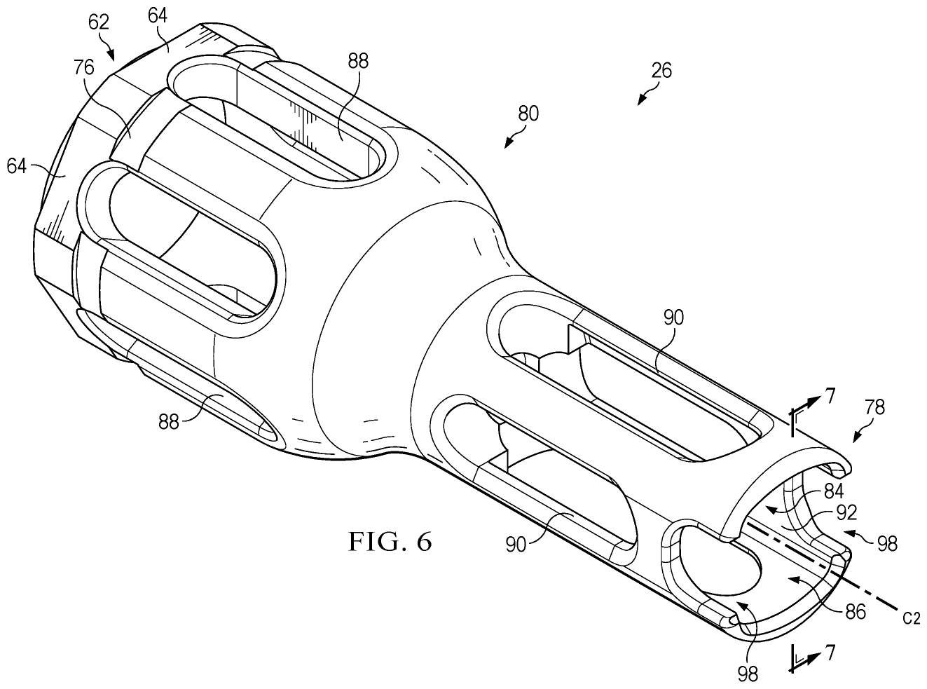

Referring now to FIGS. 5 and 6, the retention tip 26 will now be described in more detail. The retention tip 26 can include a distal tip 78 and a central portion 80 that is disposed between the proximal end 62 and the distal tip 78. The retention tip 26 can define an opening 82 at the proximal end 62 (FIG. 5), an opening 84 at the distal tip 78 (FIG. 6), and a passageway 86 that extends through the retention tip 26 between the openings 82, 84 for accommodating the driver bit 22. The retention tip 26 can define a centerline C2 that is central to the openings 82, 84 and the passageway 86. When the retention tip 26 is installed in the coupling sleeve 24, the centerline C2 can be coaxial with the centerline C1. The retention tip 26 can define a plurality of windows 88, 90 at the central portion 80 and the distal tip 78, respectively. A user can view the driver bit 22 through the windows 88, 90 when the driver bit 22 is being positioning onto the set screw 27 to ensure that the driver bit 22 is properly aligned and mates appropriately with the set screw 27. The windows 90 can also be large enough to allow for the set screw 27 to be installed over the tulip head 28 as will be described in further detail below.

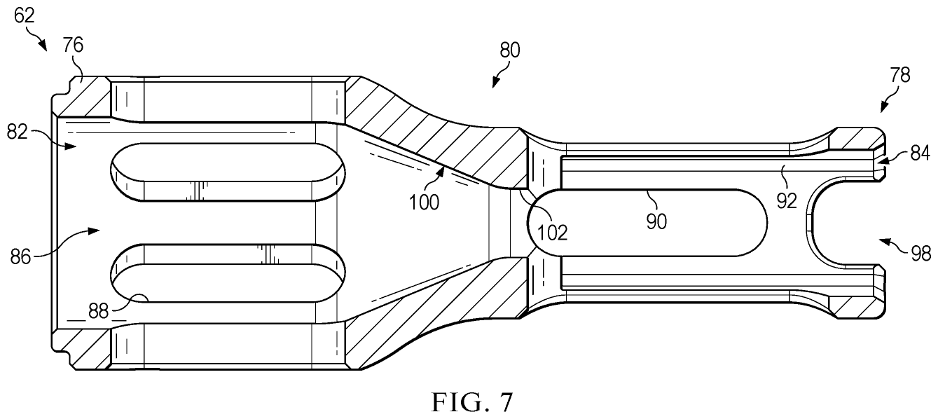

Referring now to FIG. 6, the distal tip 78 of the retention tip 26 can be configured to retain the tulip head 28 so that the set screw 27 can be properly aligned with the tulip head 28 and the driver bit 22. The distal tip 78 can include an interior wall 92 that extends to the opening 84 and is substantially rectangular shaped but with rounded corners. The tulip head 28 (FIG. 1) can include an exterior wall 96 that has a similar shape as the interior wall 92 of the distal tip 78. As such, the interior and exterior walls 92, 96 can interface with each other to retain the tulip head 28 within the distal tip 78 (via an interference fit) while also preventing rotation of the tulip head 28 relative to the retention tip 26. In one embodiment, the distal tip 78 can define a pair of notches 98 that allow for part of the tulip head 28 to be exposed at the distal tip 78 for easier manual grasping during installation of the tulip head 28 onto the distal tip 78 as well as to provide clearance to straddle an associated spinal rod when fully installed. Referring now to FIG. 7, the central portion 80 can include an interior funnel 100 that tapers from the proximal end 62 to the distal tip 78 and defines an opening 102. The interior funnel 100 can serve as a guide that directs the driver bit 22 into alignment over the set screw 27 during installation of the driver bit 22 onto the set screw 27. The retention tip 26 can be formed as a unitary one-piece construction, such as, for example, through a molding (e.g., casting), extrusion, machining, or additive manufacturing process. In one embodiment, the retention tip 26 can be formed of stainless steel but any of a variety of suitable alternative materials are also contemplated.

It is to be appreciated that although the set screw 27 is described above to be associated with a tulip head 28 and a pedicle screw 29, any of a variety of suitable alternative bone anchors that have a set screw are contemplated for use with the coupling sleeve 24. Examples of other bone anchors that have set screws are disclosed in U.S. Pat. Nos. 9,820,782; 9,844,398; and 10,729,472, which are each incorporated by reference herein in their respective entireties. Each different type of bone anchor can have a specific retention tip that is tailored to the particular bone anchor and thus enables the set screw of the bone anchor to be driven by the bone tool and corresponding driver bit. Each different retention tip can be configured to fit on the distal collar 38 of the coupling sleeve 24 in a similar manner as described above with respect to the retention tip 26. The different retention tips can accordingly be easily substituted for each other such that the coupling sleeve 24 can provide a modular arrangement that can accommodate a wide variety of different retention tips and bone anchors.

When the torque limiting attachment 20, the coupling assembly 23, and the tulip head 28 are assembled together, as illustrated in FIG. 2, the coupling sleeve 24 and the retention tip 26 can cooperate to serve as a guide that aligns the driver bit 22 with respect to the set screw 27 to ensure proper engagement therebetween for driving the set screw 27 into the tulip head 28. The coupling sleeve 24 can also facilitate rotatable coupling between the stationary collar 34 of the torque limiting attachment 20 and the tulip head 28 (via the retention tip 26). As such, when the bone power tool experiences a surge in rotational torque that causes the stationary collar 34 to rotate, such as when the bone power tool is initially activated or experiences a kick back (e.g., a counter rotational force), the coupling sleeve 24 can prevent the tulip head 28 from rotating relative to the stationary collar 34 which, during implantation of the set screw 27, can enhance the ability of the driver bit 22 to remain engaged with the set screw 27. This can result in more efficient, effective and consistent installation of the set screw 27 into the tulip head 28 than conventional arrangements that typically rely on a user's ability to manually maintain the alignment of the driver bit 22 on the set screw 27 while also adjusting for any surges in rotational torque during installation to prevent the driver bit from disengaging from the set screw 27 and possibly stripping its head.

The assembly and operation of the torque limiting attachment 20, the driver bit 22, the coupling sleeve 24, the retention tip 26, and the tulip head 28 will now be described. First, a plurality of the pedicle screws 29 that each have the tulip heads 28 affixed thereto can be implanted at different locations along a patient's spine. A support rod (not shown) can then be routed through each of the tulip heads 28. A set screw (e.g., 27) can then be preliminarily installed on each tulip head 28 such that each set screw is partially threaded into the threaded interface 30. The retention tip 26 can then be installed on the distal collar 38 of the coupling sleeve 24. The bone power tool can then be installed onto the coupling sleeve 24 by guiding the driver bit 22 through the passageways 46, 86 (e.g., along the centerlines C1 and C2) and into the opening 102 of the interior funnel 100. Such installation of the bone power tool onto the coupling sleeve 24 can also cause the inner splines 50 of the coupling sleeve 24 to mate with the outer splines 52 of the stationary collar 34 of the torque limiting attachment 20. The distal tip 78 can then be installed onto one of the tulip heads 28 such that the retention tip 26 and the coupling sleeve 24 are disposed vertically above the tulip head 28 and the pedicle screw 29. The bone power tool can then be further manipulated to encourage the driver bit 22 into engagement with the set screw 27. Once the bone power tool is properly installed on the coupling sleeve 24 and the driver bit 22 is engaged with the set screw 27, the bone power tool can be activated to drive the set screw 27 into the threaded interface 30 of the tulip head 28. As the set screw 27 is threaded into the tulip head 28, the stationary collar 34 can slide axially relative to the coupling sleeve 24 (e.g., along the centerlines C1 and C2) while also preventing rotation therebetween to allow the driver bit 22 to remain engaged with the set screw 27. Once the set screw 27 is installed on the tulip head 28, the coupling sleeve 24 and the retention tip 26 can be pulled away from the tulip head 28 and installed on another tulip head to facilitate attachment of a set screw thereto in a similar manner as described above. This process can be repeated until all of the set screws have been installed on the tulip heads associated with a support rod to facilitate securement of the support rod to all of the pedicle screws. It is to be appreciated that although the coupling sleeve 24 and the retention tip 26 have been described as being operable to install the set screw 27 on the tulip head 28, they can also be operable to facilitate removal of the set screw 27 from the tulip head 28, when appropriate. It is also to be appreciated that, in an alternative embodiment, the distal tip 78 can be configured to engage with the support rod (in addition to or in lieu of engaging the tulip head 28) when installed over the pedicle screw 29. In such an embodiment, the retention tip 26 can interface directly with the support rod to prevent rotation of the coupling sleeve 24 and the retention tip 26 relative to the support rod when driving the pedicle screw 29 with the bone power tool.

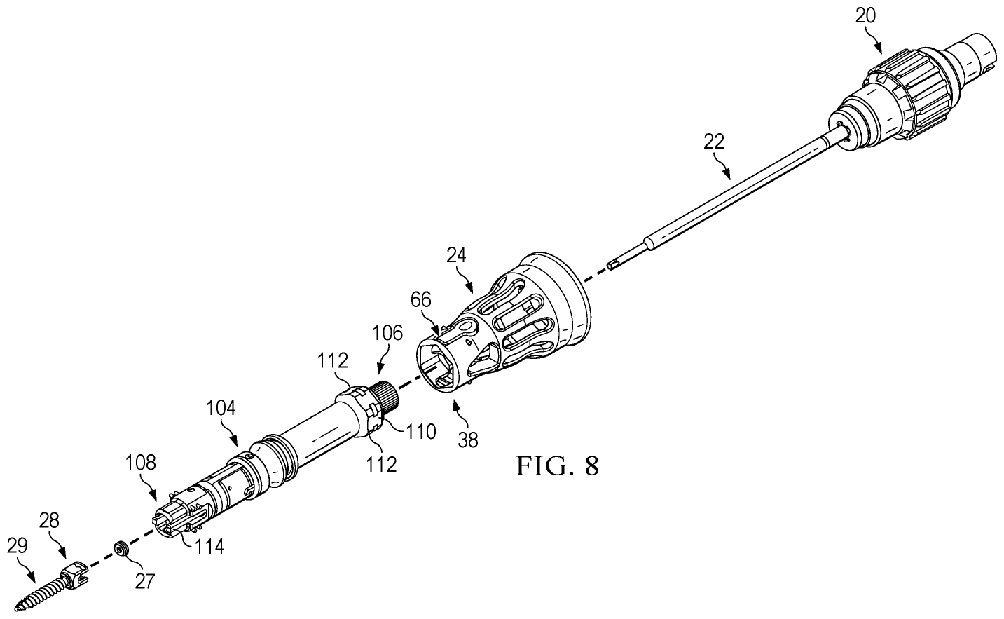

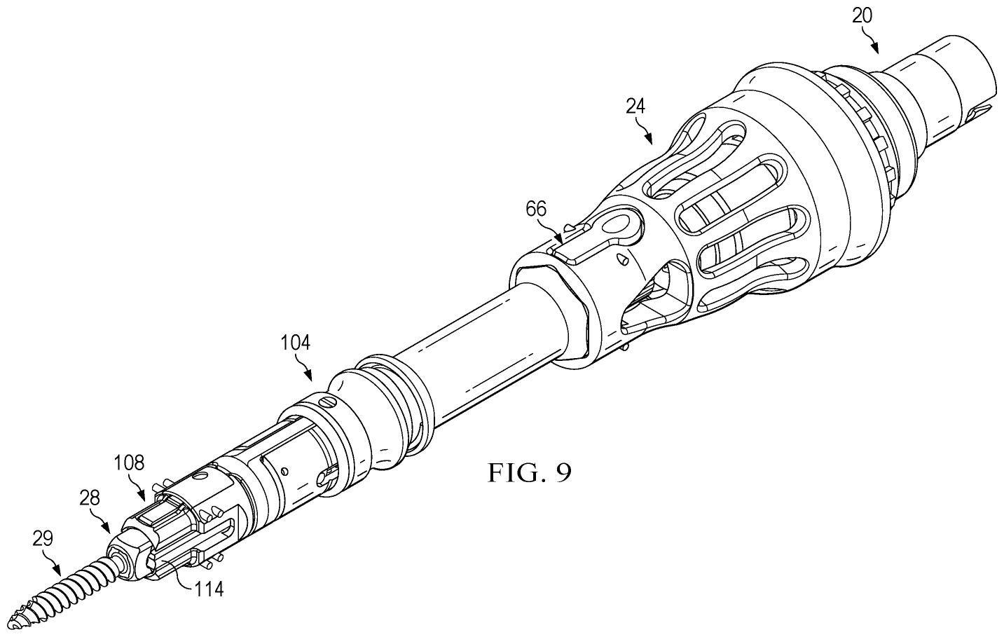

FIGS. 8 and 9 illustrate an alternative embodiment where the torque limiting attachment 20, the driver bit 22, and the coupling sleeve 24 each include all of the same features as described above with respect to FIGS. 1-4 but are associated with a rod reducer 104, instead of a retention tip (e.g., 26), that engages the tulip head 28 to facilitate attachment of the set screw 27 to the tulip head 28. It is to be appreciated that the rod reducer 104 can be used in lieu of the retention tip 26 in situations where the retention tip 26 is ineffective to reach the tulip head 28 and pedicle screw 29. As illustrated in FIG. 8, the rod reducer 104 can include a proximal end 106 and a distal end 108. The proximal end 106 can include an outer wall 110 that is substantially hexagonal shaped and can thus mate with the interior wall 60 (FIG. 4) of the distal collar 38 of the coupling sleeve 24 to facilitate coupling therebetween.

The proximal end 106 of the rod reducer 104 can define a groove 112 that interacts with the grasping members 66 to selectively couple the rod reducer 104 to the distal collar 38 of the coupling sleeve 24. The distal end 108 of the rod reducer 104 can include jaw members 114 that interface with the tulip head 28 to selectively secure the tulip head 28 with respect to the distal end 108. The rod reducer 104 can define a passageway (not shown) that allows the driver bit 22 to extend through the rod reducer 104 and into engagement with the set screw 27. When the torque limiting attachment 20, the driver bit 22, the coupling sleeve 24, the tulip head 28, and the rod reducer 104 are assembled together, as illustrated in FIG. 9, the driver bit 22 can interface with the set screw 27 in a similar manner as described above to facilitate installment of the set screw 27 onto the tulip head 28. It is to be appreciated that examples of other types of rod reducers that can be used with the coupling sleeve 24 are disclosed in PCT Pub. No. WO2022/056025 which is incorporated by reference herein in its entirety.

The foregoing description of embodiments and examples has been presented for purposes of illustration and description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent devices by those of ordinary skill in the art. Rather, it is hereby intended that the scope be defined by the claims appended hereto. Also, for any methods claimed and/or described, regardless of whether the method is described in conjunction with a flow diagram, it should be understood that unless otherwise specified or required by context, any explicit or implicit ordering of steps performed in the execution of a method does not imply that those steps must be performed in the order presented and may be performed in a different order or in parallel.

What is claimed is:

1. A coupling sleeve for a bone power tool, the coupling sleeve comprising:
a proximal collar defining a first opening that is configured to interface with a bone power tool, the first opening having a first cross-sectional shape and a first diameter;
a distal collar defining a second opening that is configured to interface with a retention feature for a pedicle screw, the second opening having a second cross-sectional shape and a second diameter;
a central portion that extends between the proximal collar and the distal collar, wherein:
the proximal collar, the distal collar, and the central portion cooperate to define a passageway between the first opening and the second opening; and
at least one of the first cross-sectional shape and the first diameter of the first opening is different from at least one of the second cross-sectional shape and the second diameter of the second opening, respectively.

2. The coupling sleeve of claim 1, wherein the first diameter is greater than the second diameter.

3. The coupling sleeve of claim 2, wherein the first diameter is at least 1.5 times greater than the second diameter.

4. The coupling sleeve of claim 1, wherein the first shape comprises a substantially cylindrical shape and the second shape comprises a substantially hexagonal shape.

5. The coupling sleeve of claim 4, wherein the first cross-sectional shape further comprises a plurality of splines.

6. The coupling sleeve of claim 1, wherein the central portion defines a window.

7. The coupling sleeve of claim 1, further comprising a grasping member configured to facilitate selective retention of the retention feature to the distal collar.

8. The coupling sleeve of claim 7, wherein the grasping member is pivotable between a latched position and a released position.

9. A coupling assembly for a bone power tool, the coupling assembly comprising:
a coupling sleeve comprising:
a proximal collar defining a first opening that is configured for mated interface with a bone power tool, the proximal collar having a first width;
a distal collar defining a second opening and having a second width that is less than the first width; and
a central portion that extends between the proximal collar and the distal collar and tapers inwardly from the proximal collar to the distal collar, wherein the proximal collar, the distal collar, and the central portion cooperate to define a passageway between the first opening and the second opening; and
a retention feature comprising:
a proximal end that is configured for insertion into the second opening to facilitate mated interaction between the proximal tip and the distal collar; and
a distal tip that is configured to support a pedicle screw.

10. The coupling assembly of claim 9, wherein:
the first opening has a first cross-sectional shape;
the second opening has a second cross-sectional shape that is different from the first cross-sectional shape; and
the proximal end of the retention feature has a third cross-sectional shape that is the same shape as the second cross-sectional shape.

11. The coupling assembly of claim 10, wherein the cross-sectional shape of each of the second opening and the proximal tip comprises a hexagonal cross-sectional shape.

12. The coupling assembly of claim 10, wherein:
the first opening has a first diameter;
the second opening has a second diameter; and
the first diameter is greater than the second diameter.

13. The coupling assembly of claim 12, wherein the first diameter is at least 1.5 times greater than the second diameter.

14. The coupling assembly of claim 9, wherein the central portion defines a window.

15. The coupling assembly of claim 9, wherein the coupling sleeve further comprises a grasping member that facilitates selective retention of the retention feature to the distal collar.

16. The coupling assembly of claim 9, wherein the retention feature comprises a retention tip.

17. The coupling assembly of claim 16, wherein:
the retention tip further comprises a central portion that extends between the proximal end and the distal tip; and
the central portion of the retention tip defines a window.

18. A coupling sleeve for a bone power tool, the coupling sleeve comprising:
a proximal collar defining a first opening that is configured to interface with a bone power tool, the first opening having a first cross-sectional shape and a first diameter;
a distal collar defining a second opening that is configured to interface with a retention feature for a pedicle screw, the second opening having a second cross-sectional shape and a second diameter;
a central portion that extends between the proximal collar and the distal collar, wherein:
the proximal collar, the distal collar, and the central portion cooperate to define a passageway between the first opening and the second opening; and
at least one of the first cross-sectional shape and the first diameter of the first opening is different from at least one of the second cross-sectional shape and the second diameter of the second opening, respectively, wherein:
the first diameter is greater than the second diameter; and
the first cross-sectional shape comprises a substantially cylindrical shape and the second cross-sectional shape comprises a substantially hexagonal shape.

19. The coupling sleeve of claim 18, wherein the first cross-sectional shape further comprises a plurality of splines.

20. The coupling sleeve of claim 18, wherein:
the proximal collar has a first width;
the distal collar has a second width that is less than the first width; and
the central portion tapers inwardly from the proximal collar to the distal collar.
